(19) United States
(12) Patent Application Publication
(10) Pub. No.: US 2014/0200662 A1
(43) Pub. Date: Jul. 17, 2014

(54) ANCHORING ELEMENTS FOR INTRACARDIAC DEVICES

(71) Applicant: MVALVE TECHNOLOGIES LTD., Herzliya (IL)

(72) Inventors: Avi Eftel, Tel-Aviv (IL); Maurice Buchbinder, La Jolla, CA (US); Shay Dubi, Tel-Aviv (IL); Amit Tubishevitz, Tel-Aviv (IL)

(21) Appl. No.: 14/156,055

(22) Filed: Jan. 15, 2014

Related U.S. Application Data
(60) Provisional application No. 61/752,994, filed on Jan. 16, 2013.
(60) Provisional application No. 61/752,996, filed on Jan. 16, 2013.
(60) Provisional application No. 61/835,588, filed on Jun. 16, 2013.

(51) Int. Cl. A61F 2/24

(52) U.S. Cl. CPC A61F 2/2409
USPC 623/2.38

(57) ABSTRACT

An intracardiac device comprising a ring-shaped body and one or more anchoring or stabilizing elements attached to said body, said elements being selected from the group consisting of levered anchoring arms, elongate anchoring arms, and lateral extension elements, wherein said device is able to move between two conformations, a collapsed conformation suitable for insertion into a delivery catheter, and an open conformation, suitable for implantation at a cardiac valve annulus.

This application claims the benefit of priority to U.S. App. No. 61/752,994 filed on Jan. 16, 2013, U.S. App. No. 61/752,996 filed on Jan. 16, 2013, and U.S. App. No. 61/835,588 filed on Jun. 16, 2013, the entire contents of each of which is incorporated by reference herein.

### FIELD OF THE INVENTION

The present invention is directed to stabilizing and anchoring elements for improving the stability of intracardiac devices. In particular, the present invention relates to the stabilization and anchoring of intracardiac devices within the mitral valve annulus.

### BACKGROUND OF THE INVENTION

Heart valve regurgitation occurs when the heart leaflets do not completely close when the heart contracts, thereby allowing blood to flow back through the improperly closed leaflets. For example, mitral valve regurgitation occurs when blood flows back through the mitral valve and into the left atrium when the ventricle contracts.

Currently, organic regurgitation is corrected by attempting to remodel the native leaflets, such as with clips, sutures, hooks, etc., to allow them to close completely when the heart contracts. When the disease is too far advanced, the entire valve needs to be replaced with a mechanical or biological prosthesis. Examples include suture annuloplasty rings, as well as actual valve replacement with leaflets, wherein the valves are sutured to the mitral valve annulus. Annuloplasty rings, which are also sutured to the annulus, have also been used to attempt to remodel the annulus, bringing the native leaflets closer together to allow them to properly close.

Based on the success of catheter-based aortic valve replacement there is growing interest in evaluating similar technologies to replace the mitral valve non-invasively using similar types of replacement valves.

Unlike the aortic valve, however, the mitral valve annulus does not provide a good landmark for positioning a replacement mitral valve. In patients needing a replacement aortic valve, the height and width of the aortic annulus are generally decreased in the presence of degenerative disease associated with calcium formation. These changes in tissue make it easier to properly secure a replacement aortic valve in place due to the reduced cross-sectional area of the aortic annulus. The degenerative changes typically found in aortic valves are not, however, present in mitral valves experiencing regurgitation, and a mitral valve annulus is therefore generally thinner, wider and softer than the annulus of a diseased aortic valve. The shape and form of the mitral valve annulus make it relatively more difficult to properly seat a replacement mitral valve in the native mitral valve annulus. The general anatomy of the mitral valve annulus also makes it more difficult to properly anchor a replacement mitral valve in place. The mitral valve annulus provides for a smoother transition from the left atrium to the left ventricle than the transition that the aortic valve annulus provides from the aorta to the left ventricle. The aortic annulus is anatomically more pronounced, providing a larger “bump” to which a replacement aortic valve can more easily be secured in place.

In general, the aortic valve annulus is smaller than the mitral valve annulus. It has been estimated that the mitral valve annulus is about 2.4 cm to about 5 cm in diameter, while the aortic valve annulus has been estimated to be about 1.6 cm to about 2.5 cm in diameter.

The larger mitral valve annulus makes it difficult to securely implant current percutaneously delivered valves in the native mitral position. Current replacement aortic valves are limited in the amount of radial expansion they can undergo during deployment and implantation. To provide a replacement aortic valve that has an expanded configuration such that it can be securely anchored in a mitral valve annulus would require that the collapsed delivery profile of the replacement aortic valve be increased. Increasing the collapsed delivery profile, however, would make endovascular delivery more dangerous for the patient and more difficult to navigate the vasculature with a larger diameter delivery system.

Various attempts have been made to deliver and implant a one-piece replacement mitral valve. However, this approach is problematic, not least because it has proven difficult to develop a device that can be collapsed down to have a sufficiently small delivery profile and still be able to be expanded and secured in place within the mitral valve via a vascular access site.

In order to overcome this problem, two-piece replacement valve systems have also been developed. Examples of a system of this type in which a two-ring structure is used to provide support for a stented valve may be found in the co-owned, co-pending international patent application that was published as WO 2012/031141. Another type of two-piece valve system, using a single-ring support structure is disclosed in the co-owned, co-pending PCT patent application having publication number WO 2013/128436

In both of the above-described prior art approaches, the valve support devices are constructed such that they may be ‘crimped’ into a collapsed delivery configuration for insertion into a delivery catheter, and then later released from said catheter. The device—which is generally constructed from a shape-memory material such as Nitinol—is then allowed to regain its former expanded working configuration prior to implantation at its working site.

One problem that needs to be addressed in relation to all of the aforementioned devices used to treat mitral valve regurgitation (and related mitral pathologies), is the need for adequate anchoring and/or stabilization in their working location close to the mitral annulus, in order to prevent their upward displacement (i.e. into the left atrium) as a result of the very strong forces generated by the left ventricle during systolic contraction on said devices.

The aim of the present invention is to provide improved stabilization and anchoring elements that may be incorporated into replacement valve support devices (such as those disclosed in the aforementioned patent applications), or into other devices intended for implantation in the region of the mitral annulus, such as annuloplasty rings and one-piece prosthetic mitral valves.

A further aim of certain embodiments of the present invention is to improve sealing around the stabilized intra-cardiac devices, thereby reducing or preventing paravalvular leakage.

Other aims and advantages of the present invention will become apparent as the description proceeds.

### SUMMARY OF THE INVENTION

The present inventors have provided technical solutions to the problem of attaining immediate (i.e. post implantation) and long-term stability of intracardiac devices. Each of these solutions is provided in the form of one or more stabilizing or anchoring elements, each of which is attached to (or forms part of) the body of the intracardiac device, which generally consists of a ring-like or annular structure defined by an outer perimeter, an inner perimeter and a central space that is bounded externally by said inner perimeter. The thickness of the device (i.e. the dimension of the device which, in use, is parallel with the longitudinal axis of the heart) is generally very small in relation to the surface area of the device that is bounded externally by the external perimeter and medially by the internal perimeter. It is to be noted that the term “ring-like” is to be understood to include annular structures that have external outlines which are either circular or non-circular (such as oval, elliptical and other regular or irregular shapes).

In particularly preferred embodiments of the present invention, the intracardiac device is a valve support device intended for use in a two-step mitral valve replacement procedure, and much of the description that follows will relate to this type of device. However, it is to be recognized that all of the various embodiments may equally apply to other types of intracardiac device, including (but not limited to) one-piece prosthetic valves and annuloplasty rings.

In the disclosure that follows, some of the structural elements of the present invention are referred to as “arms” or “wings”. It is to be noted, in this regard, that these terms are used interchangeably. Similarly the terms “stabilizing” and “anchoring” (and derivatives of said terms) are used interchangeably, when applied to the arms or wings.

Thus in a first aspect, the present invention is directed to an intracardiac device comprising a ring-shaped body, one or more anchoring arms and one or more fulcrums, wherein said anchoring arms may be caused to pivot around said fulcrums, and wherein said device is able to move between two conformations, a collapsed conformation suitable for insertion into a delivery catheter, and an open conformation, suitable for implantation at a cardiac valve annulus.

The aforementioned anchoring arms and fulcrums are mutually disposed such that each anchoring arm is capable of being pivoted in a superior-lateral direction about its fulcrum upon application of a radially-outward force to said anchoring arm.

The above-described pivotable structure acts as a lever and thus is able to apply significantly greater forces onto the ventricular wall than would be possible if the anchoring arm were constructed as an essentially static structure. In this way, the device of the present invention is capable of applying forces of sufficient magnitude to the ventricle, thereby being able to resist the strong displacement forces generated during ventricular systole. However, in order for lever structure of the present invention to function in its intended way, it is necessary to solve two further technical problems: firstly, the need to apply a sufficiently large force to the anchoring arms in order to cause them to pivot laterally and, in turn, exert similarly large forces on the inner ventricular wall, and secondly the need to time the generation of this laterally-directed expansive force such that said force is applied only when needed—that is, during the second stage of the two-stage replacement valve implantation procedure. In order to explain this second point, it is necessary to briefly consider the manner in which the mitral valve normally functions. Thus, during early systole, the intra-ventricular pressure increases to a point such that the forces that are thereby exerted on the mitral valve leaflets are sufficient to cause them to close, thereby preventing retrograde flow of blood from the left ventricle into the left atrium. During the first stage of the two-stage procedure (implantation of the support structure), the support device is not subjected to strong displacing forces during ventricular systole, due to the closure of the native leaflets, resulting in complete (or near-complete) separation between the left ventricle and left atrium. Furthermore, even when the native leaflets are open (during diastole) the valve support device is subjected to only very low pressure since firstly, the surface area of the ring-shaped support device is small, and secondly, most of said surface area is not situated in the path of the fluid flow between the atrium and ventricle. However, during the second stage of the two-stage method (implantation and expansion of the replacement valve), the native mitral valve leaflets become displaced laterally. Said leaflets are, in this way, prevented from closing during early systole. Consequently, during ventricular systole, very strong upwardly-directed forces are exerted on the replacement valve leaflets, thereby causing them to close. Since the replacement valve leaflets now form a single structure, together with the valve support device, the forces acting on said replacement valve leaflets would cause displacement of the attached valve support device were it not strongly anchored to the ventricular wall. Thus, it is during this second stage of the implantation procedure that it is essential that the anchoring arms of the support device are able to strongly engage with the ventricular wall, in order to counter the sudden increases in the displacing forces applied to said device.

The two aforementioned technical problems have been solved in an ingenious manner by the present inventors by means of exploiting the radial expansion of the replacement valve (during the second stage of the two-stage implementation method; see, for example, the methods described in co-owned WO 2012/031141 and WO 2013/128436, both of which are incorporated herein by reference), either by means of an inflatable balloon or by the use of a self-expandable stent. In this way, the outwardly-radial forces exerted by the balloon (or self-expanding stent) are transferred to the medial portion of each of the anchoring arms in the support device. Said arms are then caused to pivot about their fulcrum (more details concerning which will be provided hereinbelow). This pivoting motion then continues until the lateral portion of each anchoring arm makes contact with the ventricular wall (or, in some embodiments, together with the medial portion, causes pinching of the native valve leaflets, or in other embodiments come in contact with the device lateral attachment wings (shown in FIG. 10—thus increasing the axial force attaching the wings to the left ventricle and increasing the anchoring force of the wings). In summary: the forces applied by the expanding replacement valve cause radial expansion of the anchoring arms. As a result of the lever arrangement of said arms, the magnitude of the radially-directed force generated by the expanding valve is amplified. An additional benefit derived from the pivotable arm arrangement is that the angle formed between the lateral extremity of each expanded anchoring arm and the tissue of the ventricular wall becomes altered, such that said arm transfers said radially-directed force to said tissue in an axial direction (that is, along the longitudinal axis of the free lateral extremity of the anchoring arm). This directional effect is highly advantageous, since the geometry of anchoring arms is such that they are able to apply greater forces on the heart wall in the direction of their longitudinal axis than if said forces were to be applied at 90 degrees to said axis. In this regard, it should be noted that the aforesaid directional effect does not require that it is the free end of the anchoring arms that make contact with the tissue. Indeed, in certain circumstances such an arrangement may prove undesirable since it may result in trauma to the ventricular tissue. Rather, it is sufficient that a short length of the terminal (i.e. lateral-most) portion of the anchoring arm is angled, thereby forming a non-traumatic base. In such an arrangement, most of the forces exerted by the ventricular wall onto the anchoring arms are still directed axially, and thus buckling of the said arms is prevented. Finally, the fact that the force-generating step is the expansion of the replacement valve results in the high-magnitude forces being applied by the anchoring arms at exactly the right moment—that is, from the moment when the native valve leaflets have become immobilized.

Preferably the anchoring arm is constructed such that it is bent at a point along its length, such that said arm may be considered to comprise a medial portion and a lateral portion, wherein said portions form an angle greater than 0 degrees between them. It is to be noted that this angle may become larger or smaller during the pivoting movement of the anchoring arms. However, in one preferred embodiment, the angle is progressively reduced to almost 0 degrees (i.e. nearly complete closure of the lateral portion on to the medial portion, as the lateral expansion continues towards its endpoint.

In one preferred embodiment, the above-disclosed intracardiac device further comprises a lower fulcrum support ring connected to the ring-shaped body by means of two or more bridging elements, wherein the fulcrum is provided by the margins of an aperture formed within each of said bridging elements, and wherein each anchoring arm passes through said aperture.

In some preferred embodiments, the lower fulcrum support ring is a wire. In other preferred embodiments the lower fulcrum support ring is an annular structure.

In most embodiments of this aspect of the invention, the anchoring arms are pivotably connected to the aforementioned fulcrum support ring.

In a second aspect, the present invention is directed to intracardiac devices having elongated anchoring arms or wings. Preferably, the intracardiac device (for example, a single-ring cardiac valve support device) comprises two or more elongated anchoring wings that are cut out of the same Nitinol disc that is used to form the intracardiac device itself. Said wings are used to anchor and stabilize the support device in its working location, by means of applying pressure to the inner ventricular wall. In most of the preferred embodiments described herein, the valve support devices comprise only two such anchoring wings. However, certain versions of the device (as described hereinbelow) may possess more than two wings.

This aspect of the invention is primarily directed to an intracardiac device comprising a ring-shaped body and elongate anchoring arms, wherein each of said arms comprises a basal section that is continuous with the inner circumference of said device and a free distal tip, and wherein said device is able to move between two conformations, a collapsed conformation suitable for insertion into a delivery catheter, and an open conformation, wherein said anchoring arms are curved away from said ring, such that the proximal portion of each of said arms is generally directed medially and/or inferiorly and then laterally and/or superiorly.

In one particularly preferred embodiment of this aspect of the invention, the curvature of the arms is such that the free distal ends thereof may be caused to move upwards upon application of a radially-outward force on said arms (for example, the force exerted by a radially-expanding replacement valve located within the central cavity of a valve support device). An example of this embodiment is depicted in FIG. 8, and discussed in more detail hereinbelow.

The anchoring wings of the present invention are longer than the stabilizing structures disclosed in the aforementioned co-owned patent applications. This increased length of the anchoring wings is advantageous, since they are able to make contact with a larger area of the ventricular wall surface, thereby resulting in improved stabilization of the support device.

In some preferred embodiments of the invention, the single ring support structure contains only two wings, spaced apart by 180 degrees +/− a few degrees. The reason for this is that generally, the wings must be aligned along the mitral valve commissure in order to prevent hindrance of the native valve function during implantation of the replacement valve.

However, despite the need for the wings to be disposed opposite each other, most of the wing designs are asymmetric—that is, the two wings are not formed exactly opposite each other (i.e. exactly 180 degree separation), in order to avoid problems during crimping of the device prior to loading it into the delivery catheter. Rather they are arranged side-by-side when the disc is in a flat conformation (before the wings are bent downwards).

In other preferred embodiments, the elongated wings are not disposed opposite each other (i.e. at a separation angle of 180 degrees, but rather are mutually separated by an angle less than 180 degrees, preferably in the range of 130-179 degrees.

In a third aspect, the present invention provides means for increasing the stabilization of intracardiac devices while simultaneously solving the problem of paravalvular leakage and improving the co-axial positioning of said devices, said means comprising two or more lateral extensions, preferably separated by approximately 180 degrees around the circumference of the device. In preferred embodiments of this aspect, the lateral extensions are attached to a replacement valve support ring (either a single ring, or an upper ring of a double ring device), which are located one opposite the other. The extensions have a surface area which essentially extends the surface area of the ring laterally, to the outer aspect of the ring (i.e. extending radially outward). The length and width of the extension in the plane of the ring (the lateral plane) are significantly larger than the thickness of the extension, that is, the dimension measured along the longitudinal plane (which is typically only the width of the wire or sheet from which the extension was made). The extensions of the invention are connected to the sides of the ring in such a way as to allow said extensions to be relatively elastic and shapeable, so that the extension conforms well to the anatomy of the left atrium. In order to achieve this result, the extension elements are not continuously connected to the external aspect of the ring along their entire length, but rather are connected to the ring only at discrete singular connection points (for example, connected only at two points, on at the front edge of said element and one at the back edge thereof), without any connection at the central part of the element. Significantly, the extensions do not connect one with the other and do not form a complete ring; this allows easier crimping and reduced delivery size, to allow trans-catheter implantation of the device of the invention.

This aspect of the invention is primarily directed to an intracardiac device comprising a ring-shaped body and one or more lateral extension elements that extend laterally from the body of said device, wherein each of said extension elements is attached to the outer circumference of said ring-shaped body at two or more discrete connection points, and wherein said device is able to move between two conformations, a collapsed conformation suitable for insertion into a delivery catheter, and an open conformation, suitable for implantation at a cardiac valve annulus.

In one preferred embodiment of this aspect of the invention, the device comprises only two lateral extensions, wherein said wings are positioned opposite each other around the circumference of the ring-shaped body.

In other preferred embodiments, the device comprises four lateral extensions, arranged around the circumference of the ring-shaped body, wherein said extensions are arranged in the form of two opposing pairs of extensions. In one particularly preferred arrangement of this embodiment, the lateral extensions in one pair are larger than the lateral extensions in the second pair.

It is to be recognized that devices having other numbers of lateral extensions (e.g. three extensions, or more than four extensions) are also included within the scope of the present invention.

In certain preferred embodiments of this aspect of the invention, adjacent lateral extensions are mutually connected, by means of connecting elements, wherein said connecting elements are located on the lateral side of the ring-shaped device body.

In most preferred embodiments, each lateral extension is attached to the ring-shaped body at two or more discrete connection points (rather than being attached in a continuous manner).

When used as part of a mitral valve support device, the lateral extensions of the invention are deployed (together with the support ring) on the atrial side of the mitral annulus, and are located above the commissures of the mitral valve, in such a way that they cover the space formed by the commissures.

When the ring support of the device is placed over the mitral annulus, on the atrial side of the mitral valve, the lateral extensions of the invention effectively cover the space formed by the commissure between the leaflets and below the ring, thus preventing leakage of blood from the left ventricle into the left atrium through this space during systole.

As disclosed above, in one preferred embodiment of this aspect of the invention, the intracardiac device may comprise four lateral extension elements. Preferably, these extension elements are angled upwards towards the atrium. These elements contribute to the stability of the device by preventing it from falling down towards the ventricle through the mitral annulus. Since the extensions are covered with biocompatible fabric—they prevent leakage of blood (preventing paravalvular leakage).

It is to be emphasized that each of the three main types of stabilizing element disclosed hereinabove (i.e. lever-operated anchors, elongated anchoring wings and lateral extensions) may be used either alone or in combination in a single intracardiac device, in order to improve the stability of said device within the cardiac valve annulus. For example, in one preferred embodiment, the intracardiac device may comprise both lever-operated anchoring wings in combination with lateral extension elements, as disclosed hereinabove and described hereinbelow in more detail.

In many preferred embodiments of the present invention, the various types of stabilization and anchoring elements (lever-operated wings, elongated wings and lateral extensions) are arranged in pairs, such that each element of the same type is disposed opposite its partner (for example, two lever-operated wings disposed at a separation angle of 180 degrees). However, in other preferred embodiments, said stabilization and anchoring elements may be disposed such that the angle between them is less than 180 degrees, preferably in the range of 130 to 179 degrees.

It is to be emphasized that the anchoring and stabilizing means and elements disclosed herein may be incorporated into any suitable intracardiac device. However, preferably, the device is selected from the group consisting of a replacement valve support device, a one-piece replacement valve and an annuloplasty ring. More preferably, the device is suitable in size and form for use at the mitral valve annulus.

Thus, in summary, the present invention is directed to an intracardiac device comprising a ring-shaped body and one or more stabilizing elements attached to said body, wherein said device is able to move between two conformations, a collapsed conformation suitable for insertion into a delivery catheter, and an open conformation, and wherein said device is suitable for implantation at a cardiac valve annulus, said stabilizing elements being selected from the group consisting of:

a) anchoring arms and one or more fulcrums, wherein said anchoring arms may be caused to pivot around said fulcrums, and;

b) elongate anchoring arms, wherein each of said arms comprises a basal section that is continuous with the inner circumference of said device and a free distal tip, wherein said anchoring arms are curved away from said ring, such that the proximal portion of each of said arms is generally directed medially and/or inferiorly and then laterally and/or superiorly; and

c) lateral extension elements that extend laterally from the body of said device, wherein each of said extension elements is attached to the outer circumference of said ring-shaped body at two or more discrete connection points.

### BRIEF DESCRIPTION OF THE DRAWINGS

FIG. 1 shows a fully-expanded valve support device fitted with a single support ring, a lower fulcrum ring and levered anchoring arms.

FIG. 2 provides a side-view of the embodiment shown in FIG. 1, when in its pre-deployed configuration.

FIG. 3 provides an enlarged view of the fulcrum point in the embodiment of the device shown in FIG. 1.

FIG. 4 illustrates another embodiment of a valve support device fitted with levered anchoring arms, in which the lower ring functions both as a fulcrum support ring and as the lower ring of a two-ring valve support device. This embodiment is shown in situ at the mitral valve annulus.

FIG. 5 depicts a fully-expanded valve support device fitted with a second implementation of the levered anchoring arms of the present invention. In this implementation, the fulcrum point for the levered arms is created only following radial expansion of a lower ring-shaped wire element.

FIG. 6 provides a side view of the implementation shown in FIG. 5, in which the stirrup-like shape of the fulcrum support ring in its pre-expanded conformation may be clearly seen.

FIG. 7 depicts a further embodiment of the implementation of the device shown in FIG. 5, comprising a series of prongs and corresponding holes in the lateral and medial portions of the levered anchoring arms which are used to grasp the native valve leaflets and to hold them in their fully-open position.

FIG. 8 provides a perspective view of a third implementation of the levered anchoring arms of the present invention, in which said arms are curved and essentially devoid of any straight portions.

FIG. 9 depicts a further embodiment of the present invention, in which a valve support device is fitted with the curved anchoring arms of the third implementation (as shown in FIG. 8) in combination with the ‘leaflet pinching’ embodiment of the second implementation (as shown in FIG. 7).

FIG. 10 illustrates a valve support device of the present invention prior to expansion of the replacement valve, wherein said device comprises both levered anchoring arms and additional short static anchoring arms.

FIG. 11 depicts the embodiment of FIG. 10, following expansion of the replacement valve.

FIG. 12 illustrates a single-ring valve support device comprising two elongated anchoring wings of the present invention. The device is shown in its pre-crimped conformation.

FIG. 13 shows the device of FIG. 12, after the anchoring wings have expanded into their working conformation.

FIG. 14 shows the device of FIGS. 12 and 13, following its implantation at the cardiac annulus.

FIG. 15 depicts a different embodiment of the present invention, in which each elongated anchoring wing has an enlarged basal section.

FIG. 16 provides an enlarged view of the basal section of the anchoring wing of device of FIG. 15, in its expanded conformation.

FIG. 17 shows a further embodiment, having anchoring wings that are broader than those present in the embodiments presented in FIGS. 12 to 16.

FIG. 18 depicts another embodiment of the present invention, in which the valve support device comprises four anchoring wings—two short wings and two long wings.

FIG. 19 illustrates yet a further embodiment of the present invention, in which the elongated anchoring wings are constructed as open (i.e. non-solid) structures.

FIG. 20 depicts another embodiment having open-work anchoring wings, in which said wings have, in their working configuration, a broad, diamond-like shape.

FIG. 21 provides a perspective view of a valve support device of the present invention comprising a circular support ring, two anchoring wings and two lateral extension elements.

FIG. 22 provides a side view of the embodiment depicted in FIG. 21, in which it may be seen that the lateral extension elements are angled in an upward direction, in relation to the support ring.

FIG. 23 is a photographic representation of one embodiment of the present invention, in which a valve support device is fitted with two lateral extension elements.

FIG. 24 is a photographic representation of a valve support structure of the present invention following ex-vivo implantation in a cadaveric heart.

FIG. 25 provides a further photograph of a device of the present invention following ex-vivo implantation in a cadaveric heart.

FIG. 26 provides a plan view of a valve support device of the present invention prior to crimping, in which said device is fitted with four lateral extensions.

FIG. 27 depicts another embodiment of the invention, in which the valve support device comprises two different-sized pairs of lateral extensions.

FIG. 28 illustrates the embodiment of FIG. 27 following expansion into its working configuration.

FIG. 29 depicts a transcatheter replacement valve fitted with lever-operated anchoring wings of the present invention.

FIG. 30 depicts an embodiment of the present invention similar to that shown in FIG. 29, but additionally comprising four lateral extensions.

FIG. 31 illustrates a transcatheter annuloplasty ring fitted with curved levered anchoring wings and four lateral extensions of the present invention.

FIG. 32 depicts an alternative embodiment, which while similar to that shown in FIG. 31, has lateral extensions separated by an angle that is substantially less than 180 degrees.

### DETAILED DESCRIPTION OF PREFERRED EMBODIMENTS

As explained hereinabove, the present invention is primarily directed to means and elements for improving the stability of intracardiac devices. In one set of preferred embodiments, said devices are cardiac valve support devices for use in two-step valve replacement procedures, preferably in the mitral position. In other preferred embodiments, the intracardiac device may be a valve support device that is intended for implantation at other positions within the heart. Furthermore, the stabilizing and anchoring elements of the present invention may also be used to increase the stability of other types of intracardiac device, such as annuloplasty rings and one-piece prosthetic valves. Thus, although the detailed description that follows relates mainly (but not exclusively) to valve support devices for use in the mitral position, the present invention also includes within its scope the presently-disclosed and claimed stabilizing and anchoring elements when incorporated in any of the other aforementioned types of intracardiac device.

The description will now proceed to set out the details of each of the three main types of anchoring/stabilizing element that are included within the scope of the present invention, namely: lever-operated anchoring arms, elongated arms and lateral extension elements.

### Lever-Operated Anchoring Element

In a first implementation of this aspect of the present invention, the valve support device comprises an upper, valve support ring connected by means of two or more bridging elements to a lower fulcrum support ring. The valve support device in this implementation further comprises two or more anchoring arms (i.e. the same number of anchoring arms as the number of bridging elements), each of which is bent at a point along its length (as explained hereinabove) thereby defining a medial anchoring arm portion and a lateral anchoring arm portion. One end of each anchoring arm is attached to the upper (i.e. valve support) ring close to the point at which one of the bridging elements is attached. The opposite extremity of each anchoring arm is unconnected to any other structure in the device. The anchoring arms are disposed such that either the medial portion or the lateral portion thereof passes laterally through an aperture in the adjacent bridging element. Although said aperture may be formed in any convenient shape, in a preferred embodiment of this aspect of the invention, the aperture is rectangular. Either the inferior side or the superior side of said aperture acts as a fulcrum about which the anchoring arm is able to pivot.

In one preferred embodiment of this implementation of the device, the fulcrum support ring is provided in the form of a thin wire (for example, a Nitinol wire having a diameter of 0.4 mm). In this embodiment, the wire “ring” is in a contracted state, and takes the form of a stirrup (rather than an open ring) prior to lateral expansion of the anchoring arms. One advantage of this contracted form is that it does not interfere with native valve leaflet function during the first step of the two-step implantation procedure. Also, the minimal surface area presented by this contracted form facilitates expansion of the stented-valve in the second step of said procedure. As the expansion of the stented-valve proceeds, the forces applied thereby onto the contracted, stirrup-shaped fulcrum support element causes said element to adopt its open ring conformation.

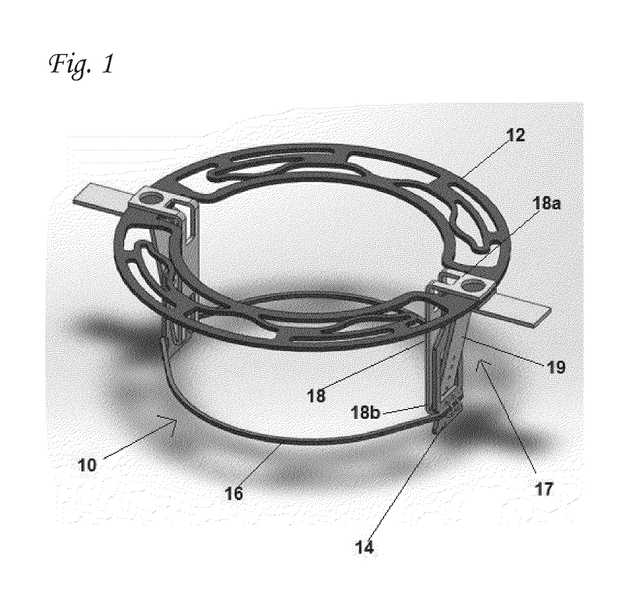

An example of this embodiment of the invention shown in its fully-expanded conformation is depicted in perspective view in FIG. 1, generally indicated as 10, which comprises an upper support ring 12, connected by two bridging elements 14 to a lower fulcrum support ring 16 which is constructed in the form of a thin Nitinol wire. The device comprises two anchoring arms 17, the medial portion 18 of each one having an upper end 18a that is attached (e.g. welded) to the upper support ring, and a lower end 18b that ends in sharply-angled portion. The lateral portion 19 of each anchoring arm then passes upwards and outwards from the angled portion, passing through a rectangular opening in bridging element 14. In the embodiment shown in this figure, the terminal portion of the distal end of lateral anchoring arm portion 19 is angled at approximately 90 degrees to the rest of said lateral portion. However, this terminal portion may also be constructed in a variety of different forms.

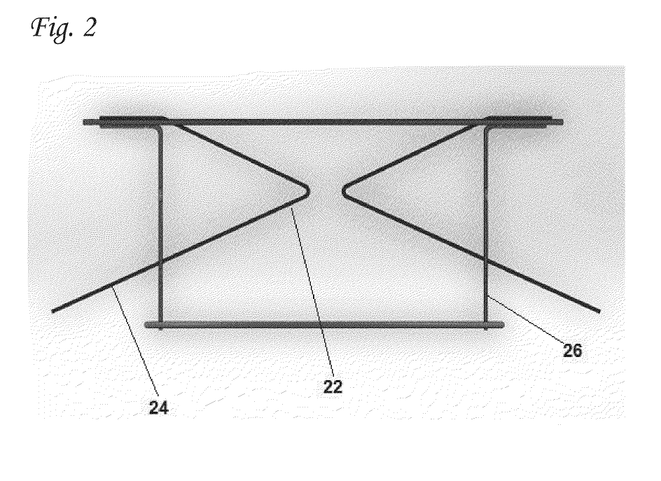

FIG. 2 provides a side-view of a device very similar to that presented in FIG. 1, but in its pre-expanded conformation. It will be seen from this figure that the angled portions 22 of each of the two anchoring arms, are initially located close to each other, within the central space of the valve support device. Then, after implantation and expansion of the replacement valve (during the second stage of the replacement procedure), the expanding valve applies pressure to the angled portions, causing them to move laterally, while each the lateral portions 24 of the anchoring arms pivots around its fulcrum point, which is provided by the lower edge of the rectangular opening in bridging element 26.

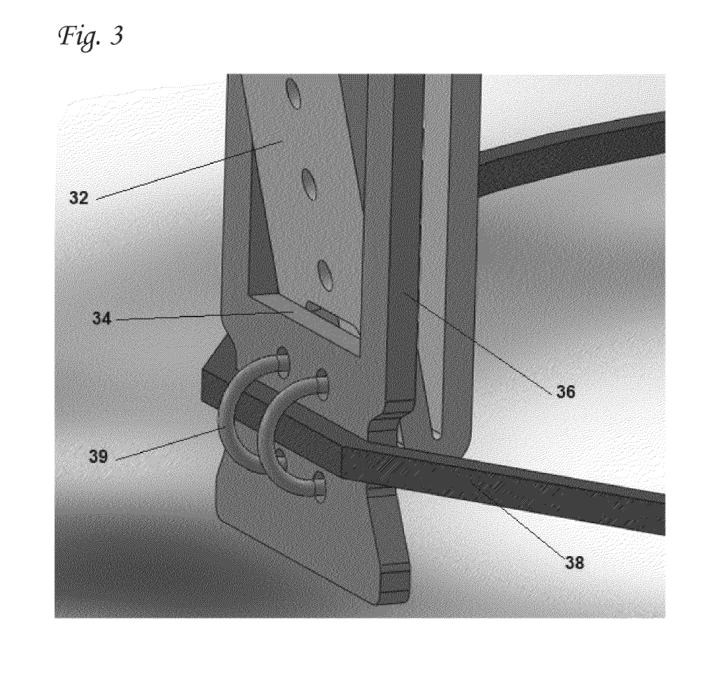

As mentioned above, in this embodiment, the lower edge of said rectangular opening acts as the fulcrum for the levered anchored arm. An enlarged view of the fulcrum point is shown in FIG. 3, in which it may be seen that the lateral portion 32 of the anchoring arm on one side of the device is in contact with—and capable of pivoting around—the lower margin 34 of the rectangular opening in bridging element 36. This figure also illustrates one way in which the bridging element 36 may be connected to the fulcrum support ring 38, namely by means of small wire staples or loops 39.

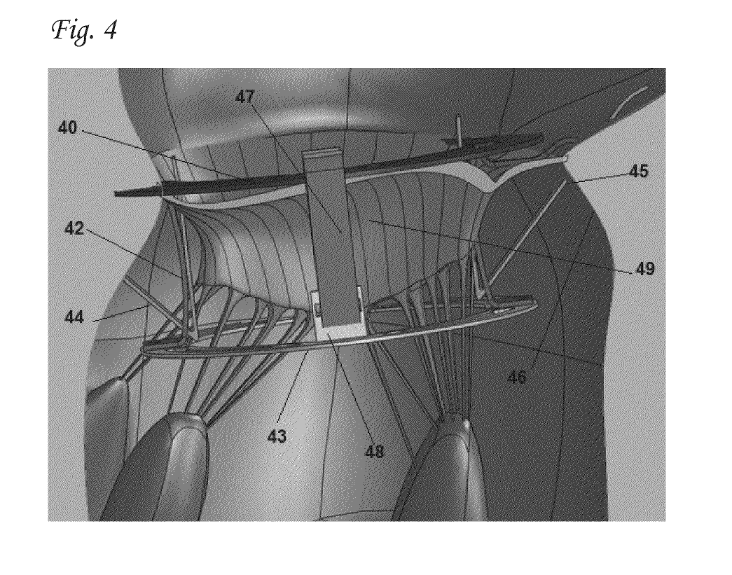

Alternatively, in other embodiments, the lower ring is cut out of a sheet of a biocompatible metal (such as Nitinol), and may function as a lower support ring of a two-ring support device (as described in co-owned, co-pending WO 2012/031141). One example of a device of this implementation having a lower support ring of this type is shown in FIG. 4, which provides a perspective in situ view of a device comprising an upper support ring 40, connected by means of bridging elements 42 to a lower support ring 43. The device is shown in its expanded conformation after implantation into the heart close to the mitral valve. The lateral portions 44 of the anchoring arms have been pushed outwards (laterally) by means of the expanded replacement valve (not shown for clarity), such that their distal tips 45 are in contact with the inner ventricular wall 46, and apply stabilizing forces thereto. While the two bridging elements 42 and associated anchoring arms are located at the ends of the mitral commissure, an additional anchoring arm 47 pivoted around a short lower fulcrum 48 is also shown. This additional anchoring arm grips the native leaflet 49, and forms part of one embodiment of the second implementation of the present invention, as will be described in more detail hereinbelow.

In a second implementation of the lever-operated anchoring wing of the present invention, the valve support device comprises an upper support ring and (similar to the first implementation) further comprises two or more anchoring arms, the superior ends of which are attached to said upper support ring. In addition, the valve support device further comprises a lower ring element that is similar to the stirrup-shaped element described in connection with one of the preferred embodiments of the first implementation, hereinabove. However, in contradistinction to the first implementation, the presently-described implementation does not comprise bridging elements connecting said stirrup-shaped element to the upper support ring. Rather, each stirrup-shaped element is connected directly to each of the anchoring arms.

Functionally, this implementation differs significantly from the first implementation described above, since when the support device is in its rest position (i.e. before radial expansion) there is no fulcrum about which the levered anchoring arms are able to rotate. Rather, the fulcrum is created only after the stirrup-shaped wire is expanded (by means of the pressure applied by the expanding stented replacement valve). At a certain point, the lower wire element becomes ring-shaped. At this point, the lower wire element is unable to expand any further, and the point of attachment of each anchoring arm to the lower wire element now functions as a fulcrum, about which said anchoring arms rotate in response to the radially-outward force generated by the expanding replacement valve. It may thus be appreciated that while in the first implementation (described above), the fulcrum is present at all stages (from pre-expansion to full expansion), there is no fulcrum in the second implementation until the lower wire element has been fully expanded into its ring conformation.

In one preferred embodiment of this implementation, the device comprises two anchoring arms which are attached to the upper support ring (and to the lower wire element) at points separated by approximately 180 degrees from each other (as measured along the circumference of the upper support ring). In this embodiment, the valve support device is intended for implantation into the mitral valve annulus such that the anchoring arms are disposed along the valve commissure such that they do not interfere with native valve leaflet function during the first stage of the two-stage implantation procedure. In addition, the lateral portions of said anchoring arms are shaped such that they may be used to apply axially-directed forces on the ventricular wall (as described above, in relation to the first implementation of the device).

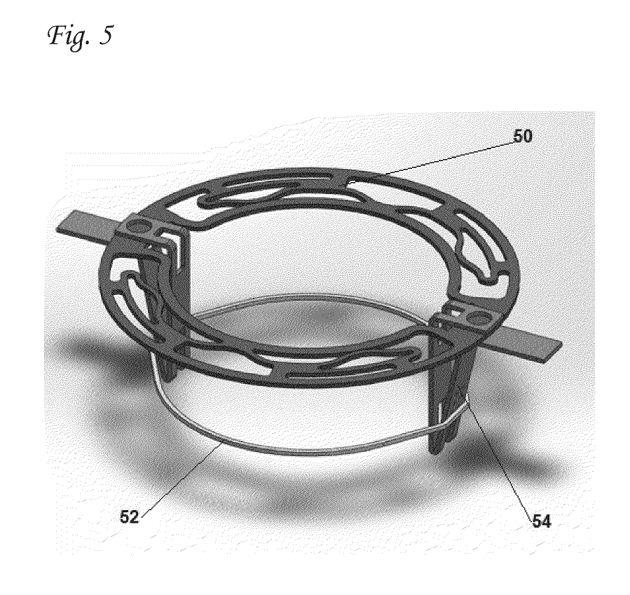

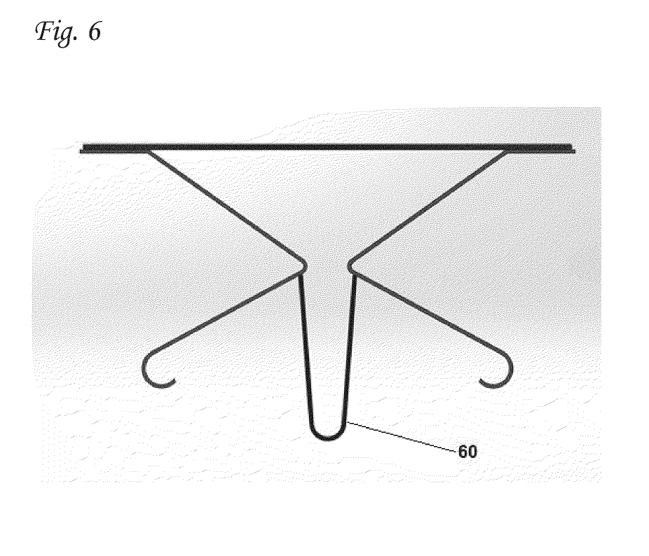

An example of a device of this type is shown in FIG. 5, which provides a perspective view of the device in its fully expanded position. As explained above, the device comprises an upper support ring 50 and a lower fulcrum support ring 52, which, in its pre-expanded conformation has a stirrup-like shape (see FIG. 6). The anchoring arms 54 are immovably attached to said upper support ring (e.g. by means of welding) and pivotably attached to lower ring/stirrup 52 by, for example, small rings or staples (not shown for clarity).

A device of this implementation, similar to that illustrated in in FIG. 5, is shown in its pre-expanded conformation in side view in FIG. 6. It may be seen from this drawing that the lower fulcrum support ring 60 is, in this conformation, stirrup-shaped and is very compact, thereby offering no resistance or interference to native valve function.

In another preferred embodiment of this implementation, the device comprises two or more anchoring arms which are constructed such that when they are in their laterally-expanded position, the angle between the medial and lateral portions of said arms is very small, such that said portions are almost in mutual contact. The small space between these portions may then be exploited in order to ‘pinch’ the native valve leaflets, thereby maintaining them in a fully-displaced, fully-open disposition. It may be appreciated that in this embodiment, anchoring and stabilizing of the support device is achieved by virtue of the fact that the anchoring arms firmly grip the valve leaflets which are in turn anchored to the ventricular wall tissues by means of the chordae tendineae and underlying papillary muscles. In one particular version of this embodiment, the leaflet-pinching effect exerted by the anchoring arms may be enhanced by the use of multiple prongs fitted to the inner surface of one of the portions (medial or lateral) of the anchoring arm, which are capable of penetrating the tissue of the entrapped valve leaflets upon lateral expansion of said arm, and becoming locked into correspondingly located and sized apertures on the inner surface of the other portion thereof.

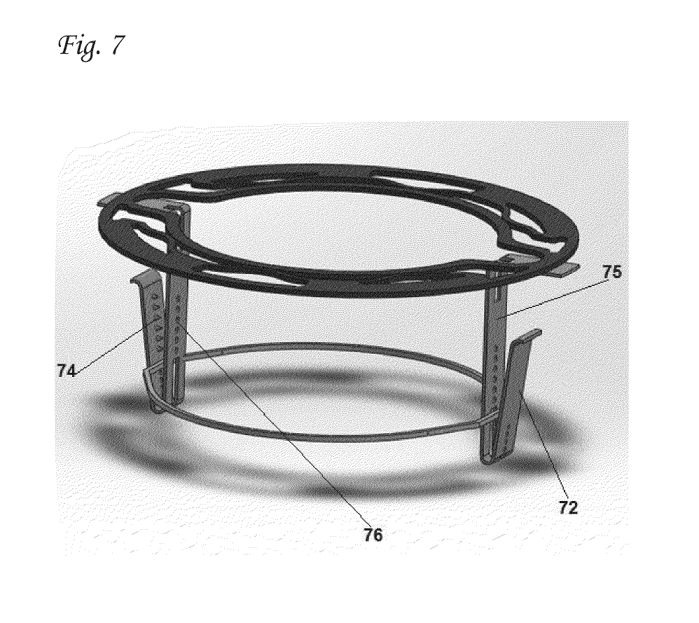

An example of this embodiment of the second implementation of the invention is shown, in perspective view, in FIG. 7. As explained hereinabove, the inner surface of one of the portions of each of the anchoring arms—in this case the lateral portion 72 is fitted with a plurality of sharp prongs 74. The medial portion 75 of each anchoring arm in this particular embodiment comprises a set of small apertures 76 which correspond in position and size with said prongs 74. In use, following expansion of the replacement valve (in the second step of the two-step replacement procedure), the lateral portion 72 of each of the anchoring arms is manipulated such that one of the native valve leaflets is trapped or ‘pinched’ between it and the medial portion 75 of the same anchoring arm, and firmly held in place by prongs 74 which penetrate the leaflet tissue and become anchored within apertures 76.

This implementation of the device of the invention thus possesses, inter alio, the following advantages:
The absence of bridging elements between the upper and lower support wings leads to a valve support structure that contains less material, and is therefore cheaper to construct, causes less interference with the native valve function and results in easier crimping of the device during its insertion into the delivery catheter.
The absence of bridging elements is further advantageous since there is now no need to align the anchoring arms (which in the first implementation were attached to said bridging elements) such that they are located along the valve commissure. Rather, the anchoring arms may (in one embodiment) be aligned such that each of the native valve leaflets becomes ‘pinched’ by the medial and lateral portions of one of the anchoring arms.
The fulcrum is created precisely when the lever effect is most needed—that is, at the point when the expanding replacement valve has caused maximum lateral displacement of the native mitral valve leaflets.

In the first two implementations of the device disclosed and described hereinabove, the support device becomes anchored to the ventricular wall only during and after expansion of the stented replacement valve. In a third implementation of the present invention, however, the valve support device comprises anchoring arms which are capable of applying both weak forces to the ventricular wall during the first stage of the two-step implantation procedure and then stronger forces during the second stage of said procedure. In order to achieve this technical effect, the device, in this implementation comprises an upper support ring to which are attached two or more curved anchoring arms that in some embodiments are essentially devoid of straight portions. In one preferred embodiment of this implementation, said curved anchoring arms initially curve in an inferio-medial direction (i.e. towards the center of the internal space of the support ring. Then, the direction of the curvature of said arms changes such that they curve inferio-laterally, laterally, superio-laterally and then in a superior direction, finally ending in a short portion that curves back inferiorly. In this particular embodiment, the curved anchoring arm has an outline form similar to an uppercase ‘D’ letter, with the flattened portion of the ‘D’ being represented by the upper portions of said arm. During the first stage of the implantation procedure, the curved arms are capable of applying relatively weak stabilizing forces to both the lateral wall of the ventricular cavity, as well as the tissue forming the roof of said cavity. Then, during and following expansion of the stented replacement valve within the central cavity of the support ring, the curved arms of said ring are pushed outwards and (as result of their curvature) upwards, such that said arms are capable of exerting much stronger forces on the lateral and superior walls of the left ventricle. In addition, the outward and upward movement of the arms changes the angle that the terminal, free portion thereof, makes with the ventricular roof, such that the forces exerted on the ventricular tissue are along the axial direction of said terminal portion (thereby preventing the buckling of the anchoring arm which may otherwise occur if the anchoring arm would meet the ventricular roof at 90 degrees to said axial direction).

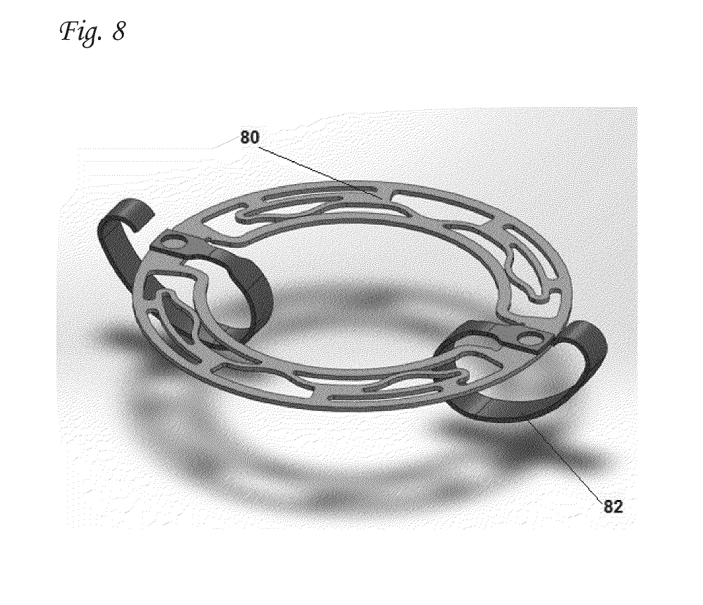

An example of this implementation of the present invention is depicted in FIG. 8. The medial ends of the curved anchoring arms 82 are attached to the support ring 80, while the lateral ends of said arms are seen to curve outwards and upwards. The device depicted in this figure is in its expanded state (i.e. following expansion of the replacement valve which would be placed within the central cavity of the support device), and the lateral ends of anchoring arms 82 are shown as if they are in a plane above the plane of support ring 80. However, in reality, said lateral ends would in fact come to rest in approximately the same plane as the support ring, and would apply strong stabilizing forces to the tissues of the ventricular roof.

It should be noted that the third implementation of the device of the present invention does not utilize levers in order to obtain a force amplification effect.

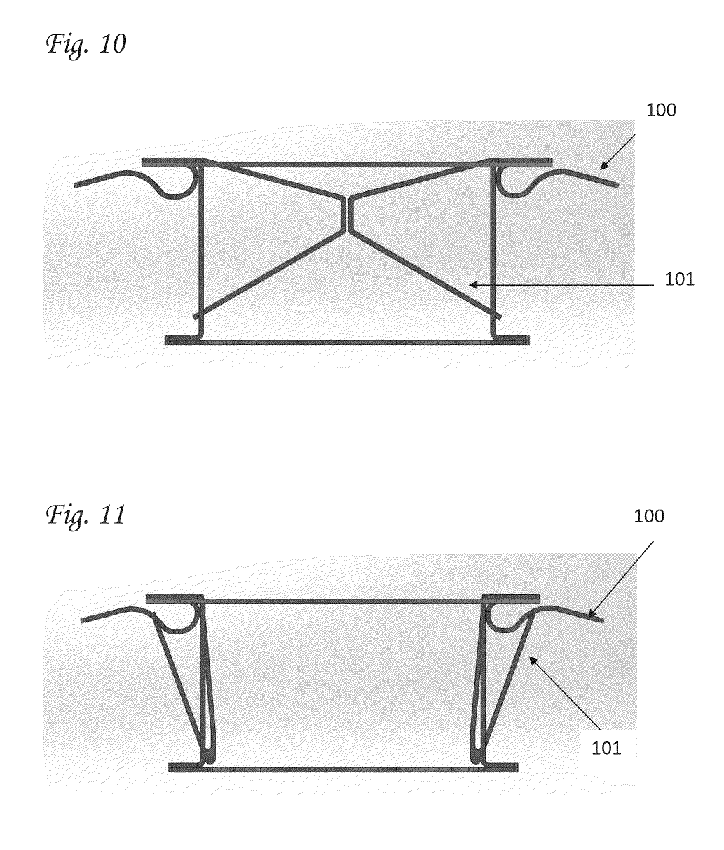

As an alternative to the third implementation of the present invention, it is also possible to construct a device comprising a combination of the first or second implementations with shorter, static anchoring arms. In such a device, the fixed arms will be used to apply relatively weak forces to the ventricular wall during the first stage of the two-stage implantation procedure, while the longer levered anchoring arms will be used to apply the stronger stabilizing forces that are required during the second stage of the procedure. In other embodiments comprising a combination of the first or second implementations with short static arms, the various anchoring elements may be arranged such that the levered anchoring arms (first or second implementations) contact the static arms (rather than ventricular tissue) during the replacement valve expansion step, thereby applying their strong stabilizing forces indirectly to the ventricular wall, that is, via the short static arms. An example of this embodiment can be seen in FIGS. 10 and 11, wherein FIG. 10 illustrates the device of the invention prior to expansion of replacement valve and FIG. 11 illustrates the device after the expansion of the replacement valve. In both figures the static arms are shown as 100 and the levered arms are shown as 101.

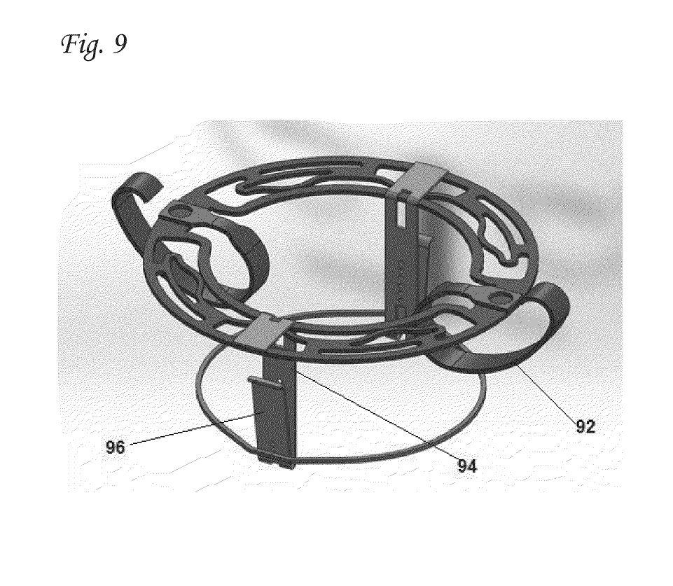

In certain other embodiments, the device may also comprise a combination of the anchoring mechanisms of several different of the above-described implementations, for example, the curved anchoring arms of the third implementation, together with the ‘leaflet pinching’ embodiment of the second implementation. Such an embodiment is shown in its expanded conformation, in perspective view, in FIG. 9. In use, the pair of curved anchoring arms 92 will be placed along the commissural line of the native mitral valve, while the medial 94 and lateral 96 portions of the levered anchoring arms will be in a position such that they can be used to entrap the native mitral valve leaflets therebetween.

In certain other embodiments of the first and second implementations of the present invention, the anchoring arms and/or bridging elements (first implementation) may additionally comprise a mechanism for locking the anchoring arms in their laterally-expanded position, such that they do not apply medially-directed forces on the replacement valve. In such an embodiment, the locking mechanism may be provided by a pin connected to the bridging element, said pin being capable of interacting with an appropriately-sized aperture formed within the levered anchoring arm.

### Elongate Anchoring Arms

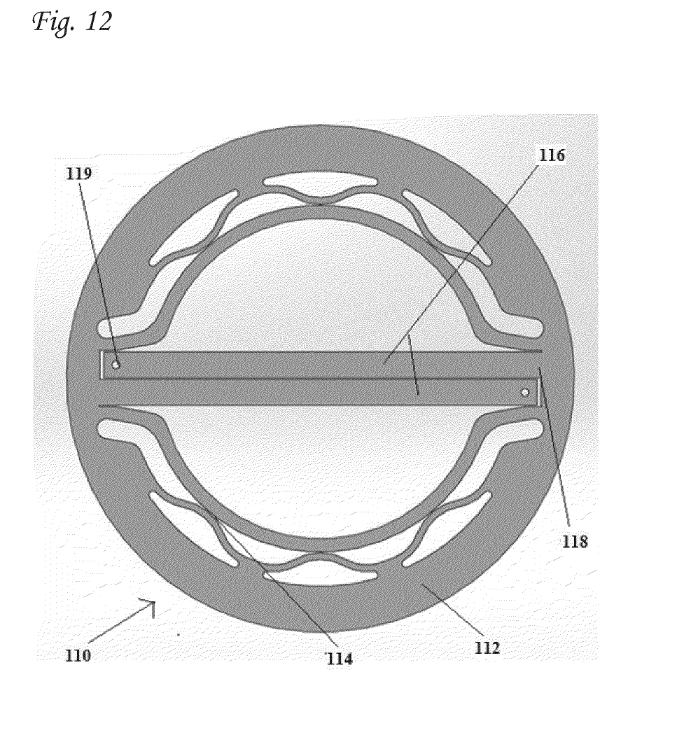

An example of a single ring support structure comprising two anchoring wings of this type is illustrated in FIG. 12. (It will be appreciated that this figure—as well as all similar figures exemplifying top views of similar devices—are intended to show said devices in their pre-crimped conformation.) The support structure 110 in this example is seen to comprise a circular support ring 112 fitted with elements 114 which permit the inner circumference of said ring to elastically deform in a radial direction (thereby facilitating the precise adaptation of the ring to a replacement valve of any size). The device also comprises two anchoring wings 116, the basal sections 118 of which are continuous with the ring itself. Indeed, in most preferred embodiments, the wings have been cut out of the same disk as the ring itself. Finally, each of said wings also has a small aperture 119 formed close to its distal tip, the purpose of said aperture being assist the operator in gripping the support device during implementation, as will described in more detail, hereinbelow.

FIG. 13 shows the same valve support device following its release from the delivery catheter, and after the anchoring wings 120 have expanded into their open, working conformation.

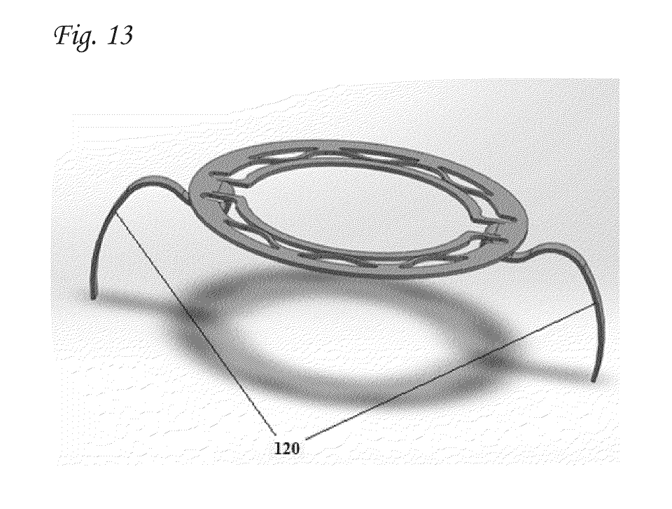

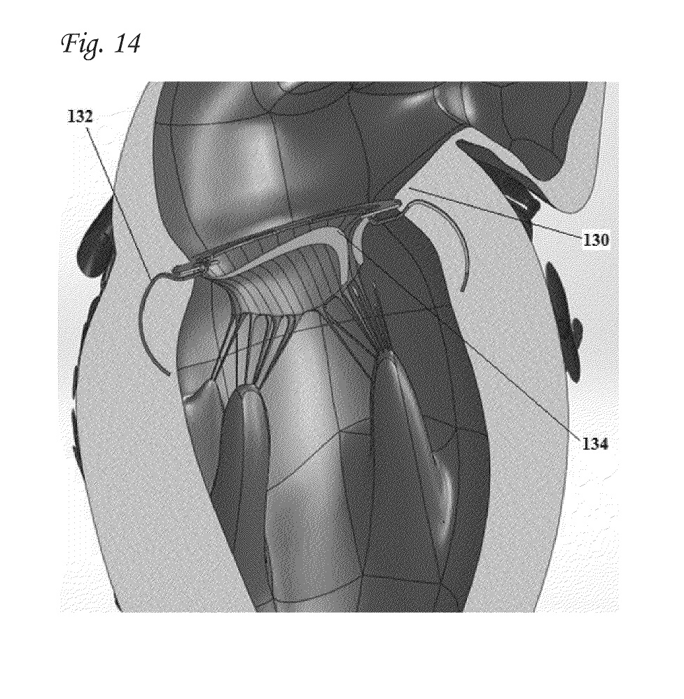

FIG. 14 illustrates the valve support device of FIGS. 12 and 13 following its implantation into the heart in the region of the cardiac annulus 130. Thus, it will be seen that the anchoring wings 132 are aligned along the commissure of native mitral valve 134, such that the presence of the support device does not interfere with the functioning of said native valve at this stage. It is to be noted that the anchoring wings 132 compress the ventricular tissue with which they are in contact, thereby causing a slight radially-outward displacement of said tissue. (This displacement is not visible in FIG. 14, due to drawing limitations.)

A different embodiment of this aspect of the invention is illustrated in FIG. 15, in which it may be seen that each anchoring wing has an enlarged basal section 140. It may be further seen in the enlarged side view of this device in its expanded conformation (shown in FIG. 16), that the expanded basal section (now shown as 150) contributes to the mechanical strength of the anchoring wing precisely at the point where said wing curves away from the ring support structure.

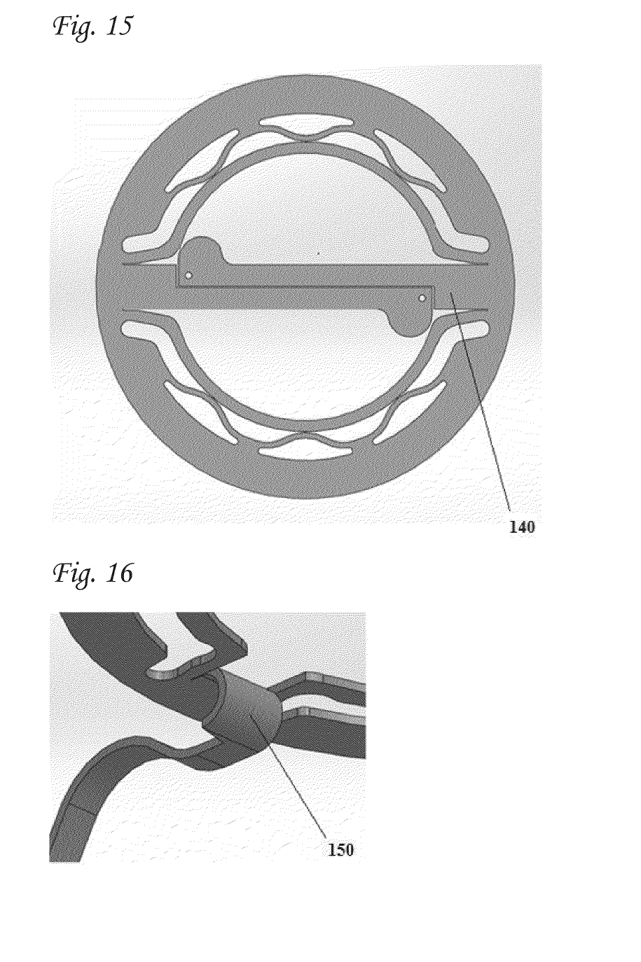

In yet another embodiment, as shown in FIG. 17, the anchoring wings 160 are broader than the wings depicted in the earlier drawings, this increased breadth being maintained along the entire length of each of said wings, from the basal section 162 to the distal tip 164. As a consequence of their greater breadth, the anchoring wings of the embodiment depicted in this figure are able to transmit a greater stabilizing force onto the ventricular tissue. This larger wing also distributes the anchoring force on a larger surface area of the heart—this is beneficial since force distribution reduces the local stress on myocardial tissue, and this may be clinically beneficial since it will prevent high stresses that may damage tissue.

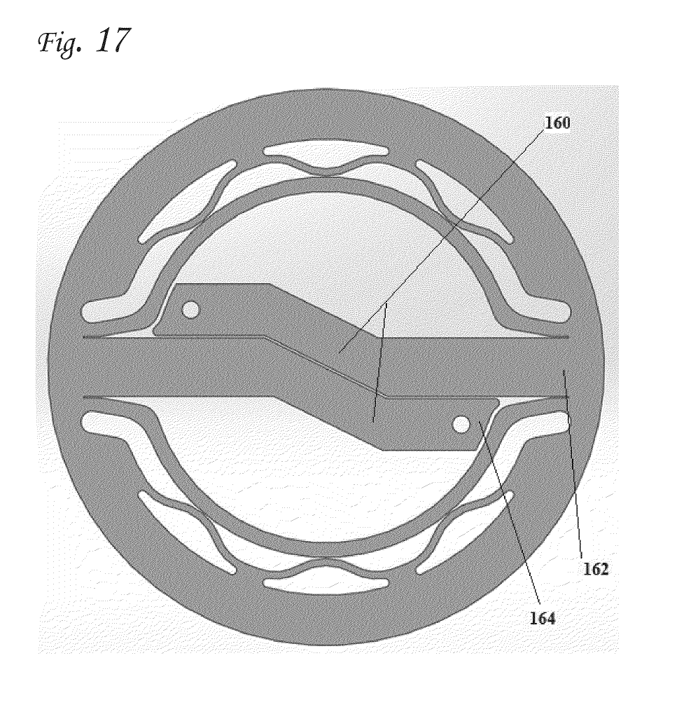

A slightly different approach is shown in FIG. 18, in which the support device comprises four anchoring wings—two short wings 170 and two long wings 172 which are disposed such that one short wing and one long wing are situated side-by-side on each side of the device. One advantage of this embodiment of the support device is that the presence of both a short wing and a longer wing on each side forms a compensatory mechanism such that in the event that one of said wings (e.g. the long wing) on each side does not make satisfactory contact with the ventricular wall, then the other one (the short wing) will be able to do so.

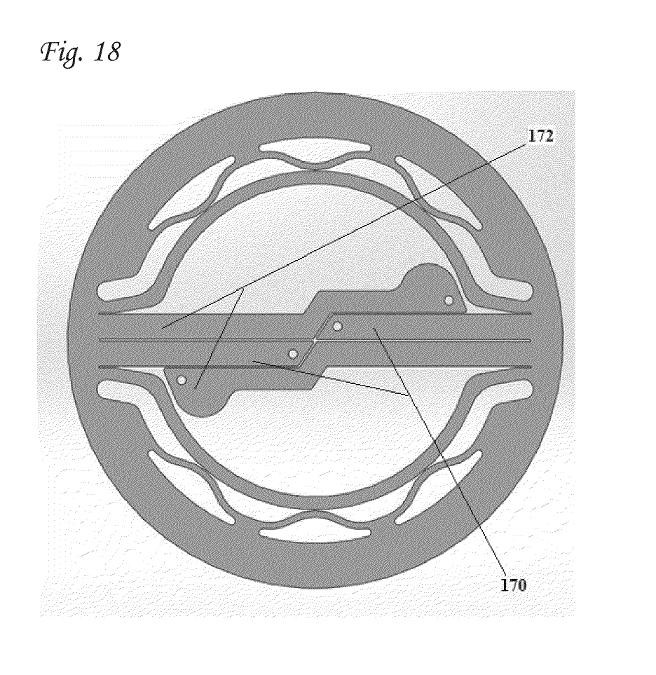

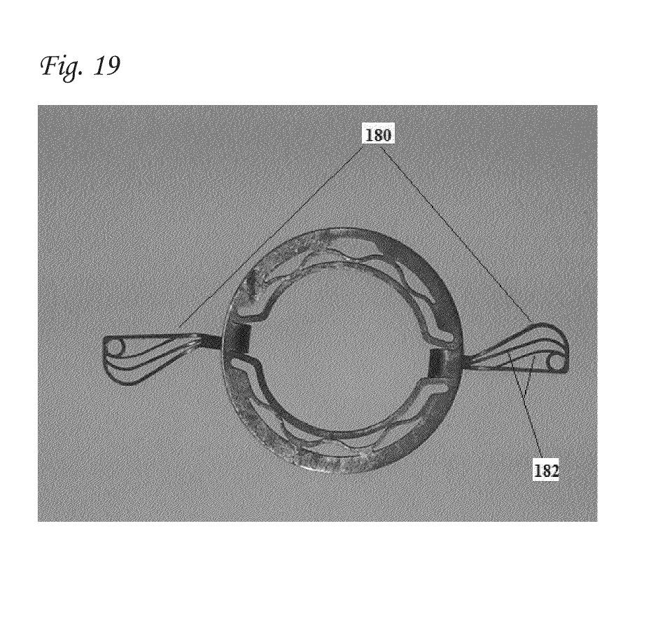

In all of the various embodiments described thus far and depicted in FIGS. 12 to 18, the anchoring wings are formed as solid structures cut out of the same disk as the support ring itself. In an alternative approach, as shown in the photographic view presented in FIG. 19, the wings 180 are constructed as open structures. This type of wing may be created, for example, by means of first cutting out a broad wing from the support ring disk, and then further removing material, such that one or more metallic strands remain within the wing. Two such strands 182 are shown in the design depicted in FIG. 19. One advantage of this approach is that broader anchoring wings may be constructed (thereby being able to apply stabilizing forces to a larger area of the ventricular wall), without adding to the bulk or weight of said wings. As previously explained, this larger wing also distributes the anchoring force on a larger surface area of the heart—this is beneficial since force distribution reduces the local stress on myocardial tissue, and this may be clinically beneficial since it will prevent high stresses that may damage tissue.

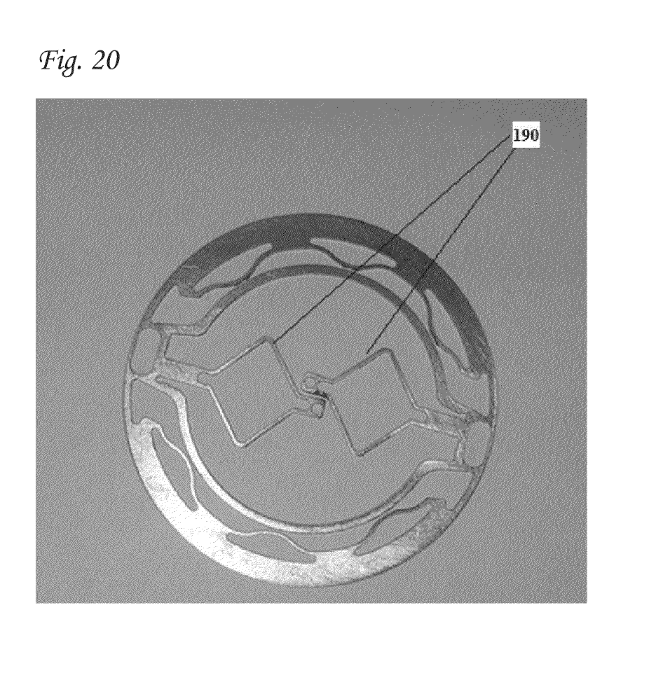

A further embodiment is shown in the photograph presented in FIG. 20. The device shown in this figure comprises wings having an open structure that are capable of existing in two different conformations—(a) an elongated, small-diameter conformation that is created during crimping during the insertion of the device into the delivery catheter and (b) a shortened, broad form, as shown in FIG. 20. As shown in the figure, the anchoring wings 190 of this specific embodiment, in their working conformation, have a broad, diamond-like shape, and are thus capable of exerting relatively high stabilizing forces on regions of the ventricular wall close to the support device. It is to be noted that if wings having this enlarged breadth were to be formed as solid structures, it would be very difficult to crimp the device into its collapsed, delivery, conformation. Thus, the use of a skeleton structure of the type shown in this figure is highly advantageous since it combines the advantages of long, narrow wings for catheter delivery with the mechanical advantages of short, broad wings once the support device has been deployed.

The wings may be covered—either completely or, alternatively, at their distal tips only—with a fabric or other covering material. In one highly preferred embodiment, a covering material, such as biocompatible Dacron, that will permit ingrowth of cardiac tissue thereinto, is used. In this way, additional anchoring of the wings to the cardiac tissue may be achieved.

The devices incorporating the elongate wings described in this section may be produced by laser cutting of Nitinol disks. The ring-ling structures that are formed thereby are then subjected to heat treatment (at temperatures of, for example, 500-600 degrees C.) with the wings bent in the desired working position, such that following release from the delivery device, the wings will adopt this new shape-memory position.

In some preferred embodiments, the wings will have small holes drilled through their distal-most portions, in order to allow the operator to easily grip the support device with a narrow-ended tool or wire during release from the delivery catheter, thereby facilitating the maneuvering of said device into its working position.

### Lateral Extension Elements

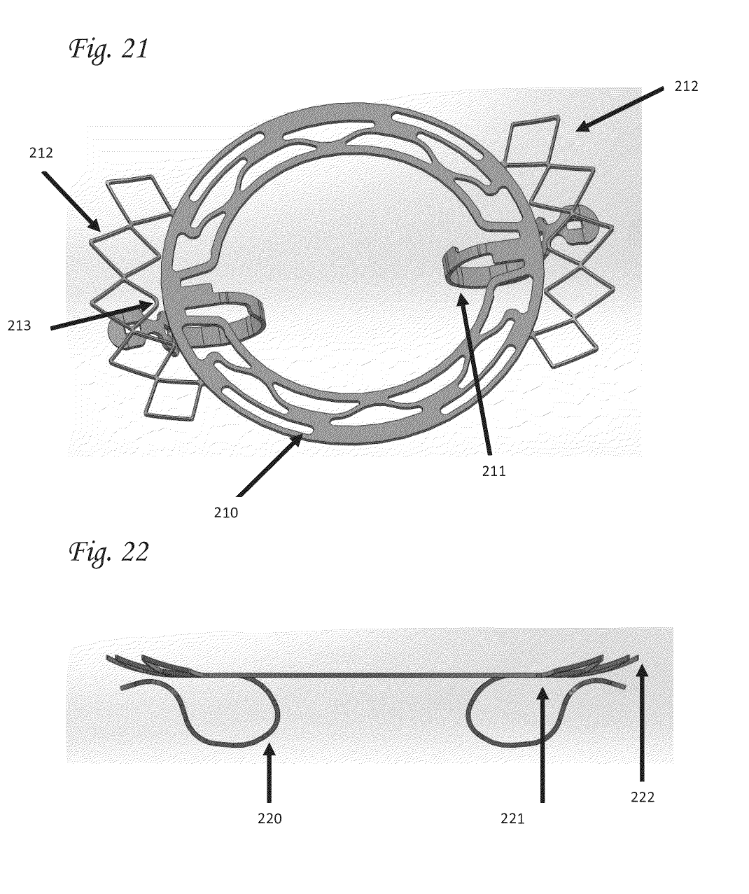

An example of a support element of the invention that comprises lateral extension elements (illustrating either a single valve support ring, or as showing only the upper ring of a double ring valve support device) is shown in FIG. 21. The support structure in this example is seen to comprise a circular support ring 210 and two exemplary anchoring wings 211, the basal sections of which are continuous with the ring itself (the anchors are exemplary only and any other anchoring and/or stabilization means may be used, as detailed in prior applications). Lateral extension elements 212 extend from two opposite sides of the ring, each is generally in an area over and covering the anchoring element of the side. The anchoring elements are located such that in deployment in a mitral annulus each anchoring element is at the area of the mitral valve commissure (between the two leaflets of the valve), and the lateral extensions of this invention are positioned such that they are located in the atria, above the area of the said commissure. This positioning allows the lateral extensions to cover the commissures, and when the lateral extensions are covered by a material (for example a biocompatible fabric such as Dacron or PTFE) which is impermeable to blood—the extension functions as a seal and reduced leakage of blood.

In another embodiment of this invention, the support device is intended for positioning in the Tricuspid valve position, which has three leaflets, and as such there may be three lateral extensions, which may similarly cover all three commissures of the valve.

In a preferred embodiment of this invention, as is shown in FIG. 21, lateral extension elements 212 are designed as a mesh structure, or a stent-like structure, in this example having deltoid shaped cells. However, this is only an example, and the shape and size of the cells and the structure of the said lateral extensions may vary, as long as it gives the function of improved sealing and positioning while being adaptable to the anatomy of the atrium.

In a preferred embodiment of this invention, as is shown in FIG. 21, the central area 213 of lateral elements 212 is not connected to the support ring. Rather, lateral extension element 212 is connected to the support ring only at the two edges of the lateral extension element, and not in the center of the element. This uniquely gives the lateral extension element an elastic ability, flexibility, which allows it to bend according to the anatomy of the atrium (and the variable anatomy of different hearts) and better fit into the atrium. An additional advantage of the flexible design is in improving the crimping ability and reducing the crimp profile of support element for trans-catheter delivery. This design for elasticity is for exemplary purpose only, and other designs, as known to the skilled artisan, are also included in the scope of this invention.

FIG. 22 is a side view of the same support element of the invention as shown in FIG. 21. The exemplary anchoring wing 220 are shown, which extend from the internal aspect of the support ring. The external aspect of the support ring, the lateral aspect, is shown as 221. Beyond the lateral aspect of the support ring 221, there is illustrated an exemplary lateral extension element of this invention, and the external, lateral, aspect of the lateral extension element is shown as 222.

In certain preferred embodiments of this aspect of the invention, the lateral extension elements are designed with an upward angle from the support ring. This angular design may assist in the anatomical positioning of the support device. However, other angles, such as downward angles (toward the ventricle), straight angle (0 degrees, extending externally at exactly the same plane as the ring), or other upward angles are included in the scope of this invention.

In a preferred embodiment of this invention, the angle between the lateral extension element and the support ring is between 0 (zero) and 30 (thirty) degrees upwards (away from the ventricle).

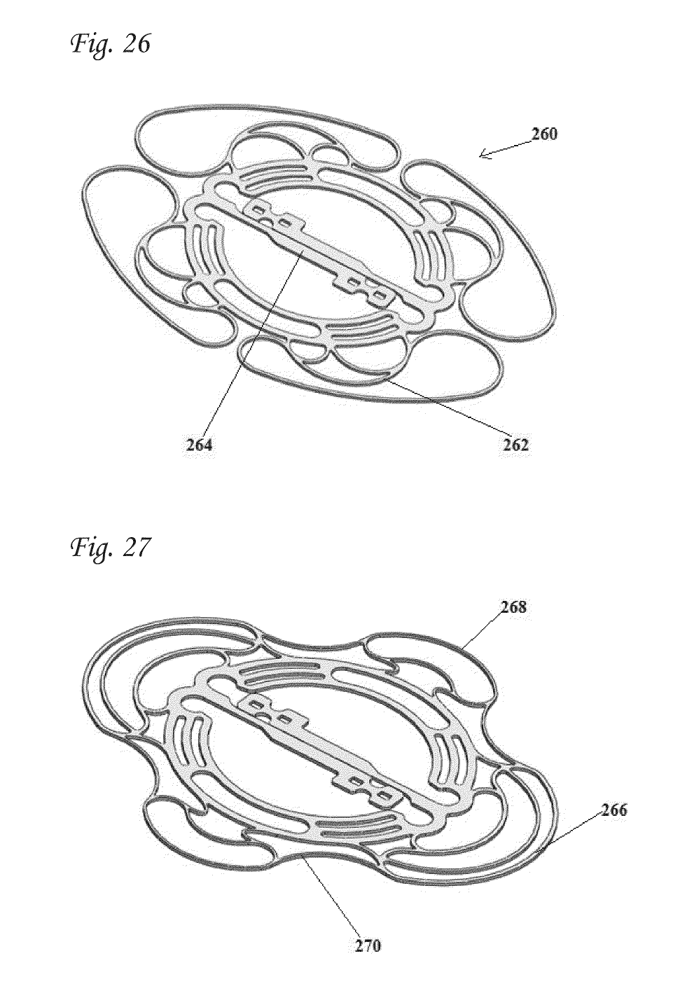

FIG. 26 shows another preferred embodiment of this aspect of the invention, in which the valve support device 260 (in its pre-crimped, flat form) comprises four separate lateral extensions 262 (as opposed to the pair of commissural lateral extension elements of the embodiments depicted in FIG. 21-25). The device also comprises a pair of elongate anchoring arms 264. The presence of lateral extension elements around the circumference of the support ring serves to prevent the device from being displaced downwards, towards the ventricle through the mitral annulus. Since the extensions are covered with a biocompatible fabric they also prevent paravalvular leakage of blood. The skeletal open-work design of the extensions provides them with suitable elasticity, such that the device may be readily crimped into a small diameter catheter (for example with a profile diameter of 18 Fr-36 Fr).

A different design comprising four lateral extension elements is shown in FIG. 27. In this embodiment, the extension elements 266 that are destined to be placed over the valve commissures are significantly larger than the anterior-posterior extensions 268. In addition, the commissural and anterior-posterior extension elements are interconnected by an optional connecting element 270. This design permits improved crimping, since the anterior-posterior extensions are smaller, thereby resulting in a smaller crimp diameter, while maintaining good sealing in the commissural area.

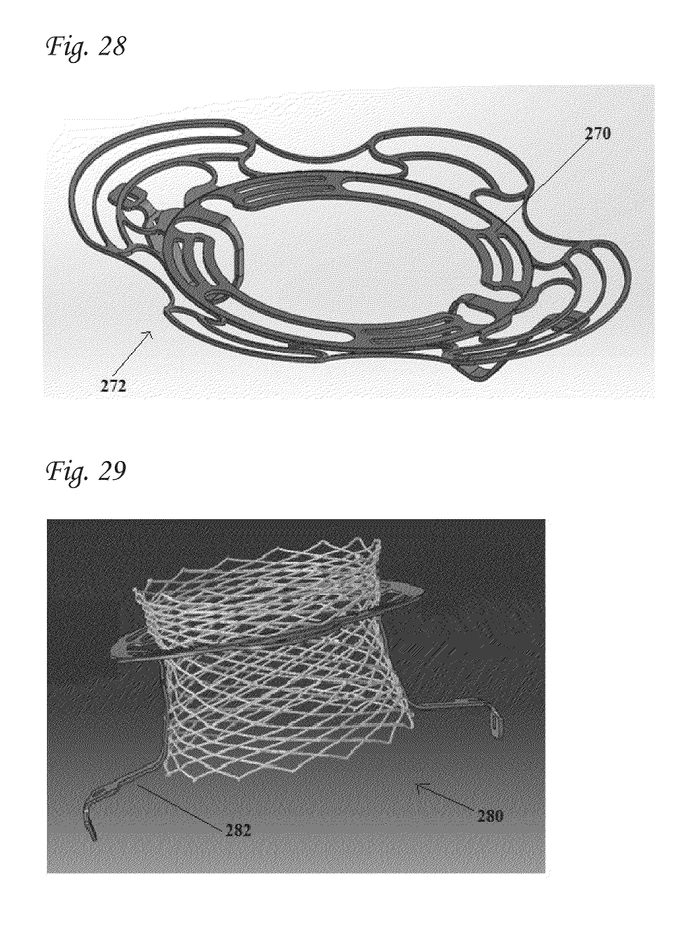

FIG. 28 shows the same embodiment as in the preceding figure, but in its expanded, working conformation. It will be seen that the four lateral extension elements together with the connecting elements are angled upwards in relation to the valve support ring 270, thereby forming a crown-like structure 272 that surrounds said ring.

Exemplary sizes of the lateral extension elements of this invention are: Radial length (length from the external aspect of the ring to the external aspect of the extension element) of 4 mm-20 mm. Circumferential length (between the two edges of the extension element) 5 mm-20 mm. Thickness of the extension element (in the longitudinal plane) of 0.2-1 mm.

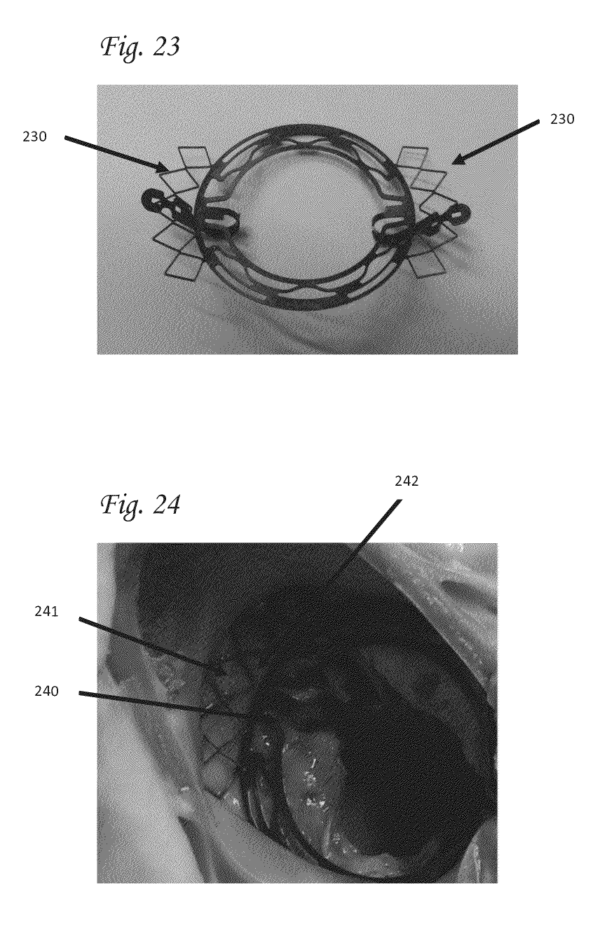

FIG. 23 is a photo of the valve support structure of the invention, demonstrating two exemplary lateral extension elements 230.

FIG. 24 is a photo of the valve support structure of the invention, implanted ex-vivo in a cadaveric heart. The support structure is located at the lower aspect of the left atrium, immediately above the mitral valve annulus. The exemplary anchoring elements 240 extend from the internal aspect of the support ring, they are directed medially and downward, between the two leaflets of the mitral valve, they extend into the left ventricle, and then extend laterally again, in the area of the commissure of the valve, thus anchoring the ring to the annulus at the area of the commissure. A lateral extension element 241 is shown, located on the wall of the left atrium. In this photo the extension element is not covered with a sealing material (and thus allows for blood flow between the cells of the element); this is for explanatory and illustration purposes only. It is emphasized in this photo with an arrow on point 242 that the lateral element is not connected to the ring in the central part of said element. Thus the connection is not continuous but partial, allowing increased flexibility of the element, and improved crimping for delivery.

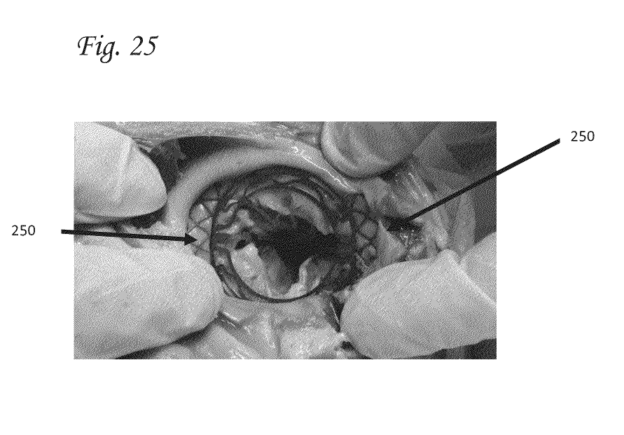

FIG. 25 is another photo of a valve support structure of the invention, implanted ex-vivo in a cadaveric heart. The support structure is located at the lower aspect of the left atrium, immediately above the mitral valve annulus. In this photo both of the lateral extension elements 250 are shown.

During deployment of the support ring on the atrial aspect of the valve, it is advantageous if the valve does not tilt (or rotate), and does not fall between the leaflets of the valve. FIG. 25 shows how the unique design of the lateral extension elements of this invention allows the support structure to be optimally located on the floor (lower aspect) of the atrium, while preventing the possibility of tilting, preventing the device from falling into the valve, and making sure the device is coaxial to the plane of the mitral valve. This allows a significant unexpected advantage in the deployment of the device, which would not be possible without the specifically designed lateral extension elements of the present invention.

The devices may be produced by laser cutting of a shape-memory material such as Nitinol, followed by heat treatment (at temperatures of, for example, 500-600 degrees C.) with the lateral extension elements bent in the desired working position, such that following release from the delivery device, the elements will adopt this new shape-memory position.

In a preferred embodiment of this invention, the lateral extension elements of this invention are covered by a material which allows sealing of said elements. Exemplary materials may be taken from the groups including biocompatible fabrics (for example Dacron, EPTFE), biocompatible plastics, nylons, etc. The elements may be either completely or partially covered by one or more of these materials.

### Other Features of the Valve Support Device of the Present Invention

A key feature of the valve support device of the present invention is the fact that it is constructed such that it may adopt two different, stable configurations: a collapsed configuration that permits the delivery of the device via a catheter that is passed through the patient's vasculature; and a second expanded configuration that the device adopts when it is caused to leave the confines of the delivery catheter during implantation within the cardiac valve annulus.

The support elements of the present invention, in their expanded configuration, generally have the form of a closed ring, the outline shape of which is preferably circular or near-circular. However, these ring elements may also be constructed in any other desired and suitable shape, such as oval, elliptical and so on.

In a particularly preferred embodiment of the present invention, the valve support device is of a size and shape that permits it to be implanted within the annulus of a mitral valve.

In preferred embodiments of the invention, the support elements are made from a resilient material that can be deformed into a delivery configuration yet are adapted to self-expand to an expanded configuration, with optional additional expansion by means of balloon dilation. For example, the support can be made from Nitinol, relying on its superelastic properties. In some embodiments the valve support is made from a material with shape memory properties, such as Nitinol, and is adapted to return to an expanded memory configuration after being heated above its transition temperature. In some embodiments in which the valve support is made from a material such as Nitinol, the shape memory properties and the superelastic properties are utilized.

In another preferred embodiment, the valve support device of the present invention may be constructed as a two-ring device, comprising an upper support element and a lower support element, wherein said support elements are mutually connected by two or more bridging elements. Examples of such an embodiment may be found in co-pending, co-owned international patent application no. PCT/US2011/050232 (published as WO 2012/031141, the entire contents of which are incorporated herein by reference). All of the structural and functional features of the anchoring wings described hereinabove in connection with the single ring support devices apply equally to this two-ring embodiment.

In the case of the two-ring support element embodiments the height of the valve support, measured from the base of the first support to the top of the second support, is generally in the range of about 2 mm to about 5 cm to be able to accommodate the height of the replacement heart valve, such as a stented heart valve. In some embodiments the height is greater than 5 cm. In some embodiments the height of the valve support is between about 1 cm and about 2.5 cm. For example, a stented heart valve in an expanded configuration can have a height of about 17.5 mm. It should be noted, of course, that these numbers are merely exemplary and are not limiting in any way.

In another aspect, the present invention also provides a two-stage method for implanting a replacement cardiac valve, wherein the first stage comprises delivering a valve support device fitted with stabilizing wings of the present invention to a location near a subject's cardiac valve; expanding the support element from a collapsed configuration to an expanded, deployed configuration secured against cardiac tissue in the region of the valve annulus, such that the presence of the expanded support element does not interfere with the function of the native valve leaflets, and further comprising moving, bending or otherwise adapting the position of said stabilizing wings such that at least the distal ends thereof are brought into contact with the inner cardiac wall; and wherein the second stage comprises securing a replacement valve to the valve support. Securing the replacement cardiac valve to the valve support can comprise expanding the replacement cardiac valve from a collapsed delivery configuration to an expanded configuration. Expanding the replacement cardiac valve can include expanding the replacement cardiac valve with a balloon and/or allowing the replacement cardiac valve to self-expand. Securing a replacement cardiac valve to the valve support can comprise securing the replacement cardiac valve radially within the valve support.

During deployment of the replacement cardiac valve, the expansion of said valve causes the lateral displacement of the native valve leaflets, thereby disabling them, the cardiac valve function now be solely fulfilled by the leaflets of the deployed replacement cardiac valve.

In a highly preferred embodiment of the method of the invention, the cardiac valve to be replaced is a mitral valve.

In one preferred embodiment of this method, the valve support device is a single-ring device of the type described hereinabove. In a further preferred embodiment of this method, the valve support device is a two-ring device comprising an upper support element and a lower support element mutually connected by two or more bridging elements. In this embodiment, the above-disclosed step of expanding the support elements comprises expanding, in sequence, one of the support elements, the bridging elements and the second support element.

In one embodiment, the above-defined method may be employed to deliver the valve support by an endovascular route. In another embodiment, the method may be used to deliver the valve support by a transapical route.

All of the components of the various embodiments of the device disclosed and described hereinabove may be constructed using any suitable biocompatible material possessing shape memory and/or superelastic properties. These properties are required in order to permit the valve support device of the invention to be transformed between a collapsed conformation (such that said device may be loaded into a delivery catheter) and an expanded, working conformation. While a preferred material for use in constructing the device is Nitinol, other suitable metallic and non-metallic materials may also be used and are included within the scope of the present invention. The various embodiments described herein may be constructed using any of the standard manufacturing techniques known to the skilled artisan in this field, including laser cutting, spot welding and so on.

While primarily intended for use in two-step mitral valve replacement procedures (such as described in detail in co-owned, co-pending international patent application WO 2012/031141 and U.S. patent application No. 61/604,083 (both of which are incorporated herein by reference), the anchoring and stabilizing elements of the present invention that are disclosed and described hereinabove may also be used in various other medical surgical procedures, particularly (but not exclusively) in the field of cardiology. As mentioned hereinabove, other exemplary intracardiac devices that may incorporate the anchoring elements of the present invention include (but are not limited to) valve support devices intended for use at sites other than the mitral valve annulus, annuloplasty rings and one-piece prosthetic valves. Examples of some of these types of intracardiac device that incorporate the stabilizing and anchoring elements of the present invention are shown in FIGS. 29-32.

Thus, FIG. 29 illustrates a one-piece transcatheter prosthetic valve 280 comprising a pair of lever-operated anchoring wings, according to the present invention.

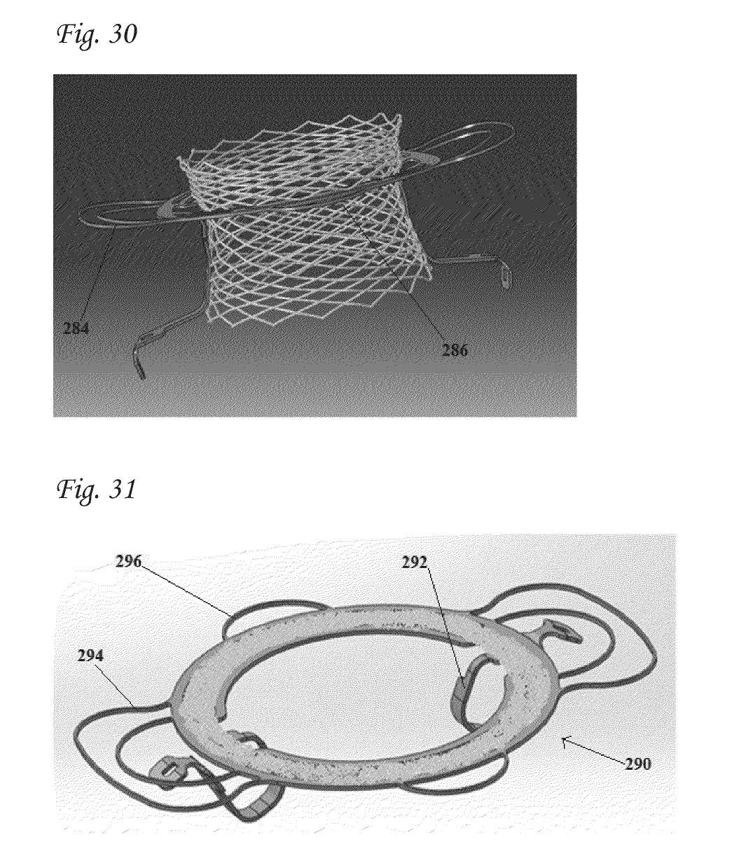

FIG. 30 shows a similar one-piece prosthetic valve to that depicted in FIG. 29, but which further comprises lateral extension elements in both the commissural position 284 and the anterior-posterior position 286, which together form a crown-like structure surrounding the valve.

FIG. 31 shows a transcatheter annuloplasty ring 290 comprising the lever-operated anchoring arms of the present invention 292, together with a lateral extension ‘crown’, consisting of two commissural lateral extension elements 294 and two anterior-posterior lateral extension elements 296.

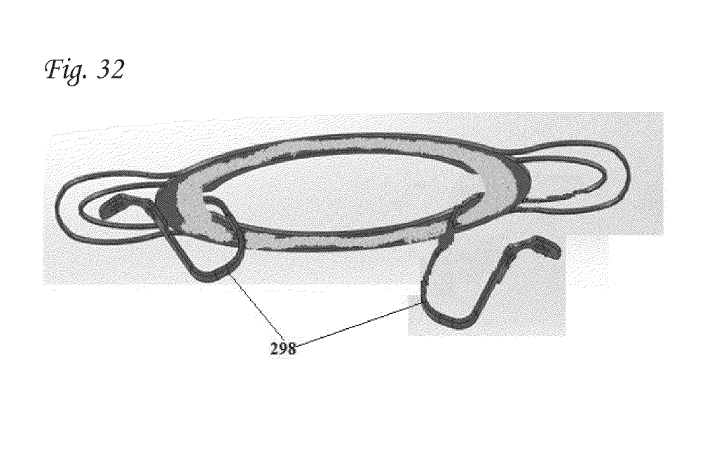

FIG. 32 depicts a similar annuloplasty ring to that shown in FIG. 31. In this embodiment, however, the lever-operated anchoring arms 298 are not placed opposite each other, but rather there is a separation angle between them of significantly less than 180 degrees.

1. An intracardiac device comprising a ring-shaped body, one or more anchoring arms and one or more fulcrums, wherein said anchoring arms may be caused to pivot around said fulcrums, and wherein said device is able to move between two conformations, a collapsed conformation suitable for insertion into a delivery catheter, and an open conformation, suitable for implantation at a cardiac valve annulus.

2. The intracardiac device according to claim 1, further comprising a lower fulcrum support ring connected to the ring-shaped body by means of two or more bridging elements, wherein the fulcrum is provided by the margins of an aperture formed within each of said bridging elements, and wherein each anchoring arm passes through said aperture.

3. The intracardiac device according to claim 2, wherein the lower fulcrum support ring is a wire.

4. The intracardiac device according to claim 2, wherein the lower fulcrum support ring is an annular structure.

5. The intracardiac device according to claim 1, wherein the anchoring arms are pivotably connected to the fulcrum support ring.

6. An intracardiac device comprising a ring-shaped body and elongate anchoring arms, wherein each of said arms comprises a basal section that is continuous with the inner circumference of said device and a free distal tip, and wherein said device is able to move between two conformations, a collapsed conformation suitable for insertion into a delivery catheter, and an open conformation, wherein said anchoring arms are curved away from said ring, such that the proximal portion of each of said arms is generally directed medially and/or inferiorly and then laterally and/or superiorly.

7. The intracardiac device according to claim 6, wherein the curvature of the arms is such that the free distal ends thereof may be caused to move upwards upon application of a radially-outward force on said arms.

8. An intracardiac device comprising a ring-shaped body and one or more lateral extension elements that extend laterally from the body of said device, wherein each of said extension elements is attached to the outer circumference of said ring-shaped body at two or more discrete connection points, and wherein said device is able to move between two conformations, a collapsed conformation suitable for insertion into a delivery catheter, and an open conformation, suitable for implantation at a cardiac valve annulus.

9. The intracardiac device of claim 8, comprising only two lateral extensions, wherein said wings are positioned opposite each other around the circumference of the ring-shaped body.

10. The intracardiac device of claim 8, comprising four lateral extensions, arranged around the circumference of the ring-shaped body, wherein said extensions are arranged in the form of two opposing pairs of extensions.

11. The intracardiac device of claim 10, wherein the lateral extensions in one pair are larger than the lateral extensions in the second pair.

12. The intracardiac device of claim 8 wherein adjacent lateral extensions are mutually connected, by means of connecting elements.

13. The intracardiac device of claim 8, wherein each lateral extension is attached to the ring-shaped body at two or more discrete connection points.

14. The intracardiac device of claim 1, wherein said device is selected from the group consisting of a replacement valve support device, a one-piece replacement valve and an annuloplasty ring.

15. The intracardiac device of claim 14, wherein the valve support device is suitable for use at the mitral valve annulus.

16. The intracardiac device of claim 14, wherein the one-piece replacement valve is suitable for use at the mitral valve annulus.

17. An intracardiac device selected from the group consisting of valve support device, one-piece prosthetic valve and annuloplasty ring, wherein said device comprises one or more of the anchoring or stabilizing means selected from the group consisting of:
one or more anchoring arms and one or more fulcrums, wherein said anchoring arms may be caused to pivot around said fulcrums, and wherein said device is able to move between two conformations, a collapsed conformation suitable for insertion into a delivery catheter, and an open conformation, suitable for implantation at a cardiac valve annulus;
one or more elongate anchoring arms, wherein each of said arms comprises a basal section that is continuous with the inner circumference of said device and a free distal tip, and wherein said device is able to move between two conformations, a collapsed conformation suitable for insertion into a delivery catheter, and an open conformation, wherein said anchoring arms are curved away from a ring-shaped body, such that the proximal portion of each of said arms is generally directed medially and/or inferiorly and then laterally and/or superiorly; and
one or more lateral extension elements that extend laterally from the body of said device, wherein each of said extension elements is attached to the outer circumference of a ring-shaped body at two or more discrete connection points, and wherein said device is able to move between two conformations, a collapsed conformation suitable for insertion into a delivery catheter, and an open conformation, suitable for implantation at a cardiac valve annulus.

18. An intracardiac device comprising a ring-shaped body and one or more stabilizing elements attached to said body, wherein said device is able to move between two conformations, a collapsed conformation suitable for insertion into a delivery catheter, and an open conformation, suitable for implantation at a cardiac valve annulus, said stabilizing elements being selected from the group consisting of:
a) anchoring arms and one or more fulcrums, wherein said anchoring arms may be caused to pivot around said fulcrums;
b) elongate anchoring arms, wherein each of said arms comprises a basal section that is continuous with the inner circumference of said device and a free distal tip, wherein said anchoring arms are curved away from said ring, such that the proximal portion of each of said arms is generally directed medially and/or inferiorly and then laterally and/or superiorly; and
c) lateral extension elements that extend laterally from the body of said device, wherein each of said extension elements is attached to the outer circumference of said ring-shaped body at two or more discrete connection points.
